(19) United States
(12) Patent Application Publication
(10) Pub. No.: US 2007/0135399 A1
(43) Pub. Date: Jun. 14, 2007

(54) Heteroaromatic sulphonamide prodrugs

(76) Inventor: Ralf Wyrwa, Rothenstein (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/605,482

(22) Filed: Nov. 29, 2006

Related U.S. Application Data
(60) Provisional application No. 60/742,523, filed on Dec. 6, 2005.

(30) Foreign Application Priority Data
Nov. 30, 2005 (DE) 10 2005 057 421.1

(51) Int. Cl. A61K 31/58; C07J 43/00; A61K 31/4745; A61K 31/4706

(52) U.S. Cl. 514/176; 540/107; 514/305; 514/313; 514/452; 546/159; 546/137

(57) ABSTRACT

The invention relates to sulphonamide prodrugs of the general formula I, 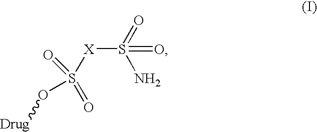 having a heteroaromatic linker, to a process for their preparation, to pharmaceutical compositions comprising these compounds and to their use for the production of orally available medicaments. The compounds according to the invention bind to carboanhydrases and inhibit these enzymes.

This application claims the benefit of the filing date of U.S. Provisional Application Ser. No. 60/742,523 filed Dec. 6, 2005.

The invention relates to sulphonamide prodrugs of the general formula (I), 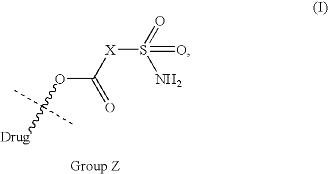
in which X is a heteroaromatic, to a process for the preparation of these prodrugs, to pharmaceutical compositions comprising these compounds and to their use for the production of orally available medicaments.

From WO 01/91797, steroidal compounds are known which are bonded to erythrocytes via a group —SO₂NR¹R² and accumulate there. The concentration ratio of the compounds between erythrocytes and plasma is 10-1000:1, preferentially 30-1000:1, such that we can speak of depot formation in the erythrocytes. Owing to the strong bonding of the compounds to the erythrocytes, metabolization during the liver passage is avoided. Disadvantageously, despite reduced metabolization using the dosages indicated, therapy-relevant active compound levels are not afforded. The reasons for this are to be sought in excessively strong binding to erythrocytes, cleavage induced by enzymes and in low solubilities.

It is the object of the invention to make available novel prodrugs which are orally available and in comparison to the prior art guarantee a therapy-relevant active compound level even at a low dosage.

This object is achieved by heterocyclic sulphonamide prodrugs of the general formula (I), in which a sulphamoyl radical is bonded to the drug to be released via a heteroaromatic spacer X by means of a carboxylic acid ester bond,
in which 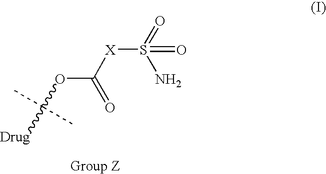
X is an unsubstituted or substituted heteroaromatic radical or an alkylheteroaromatic and,
Drug is a pharmaceutical active compound which can form a carboxylic acid ester by means of an OH group, such as steroids, anti-malarial agents, nucleosides, isoflavanoids, which can optionally be substituted.

The sulphonamide prodrugs according to the invention having a heteroaromatic linker X bind to erythrocytes, are readily water-soluble and are cleaved hydro-lytically without the participation of enzymes.

In the sense of the invention, a heteroaromatic radical is, for example, thiophene, pyridine, pyrrole, furan or alternatively thiophene, pyridine, pyrrole or furan substituted by C₁₋₄-alkyl or halogen. For substituted heteroaromatics, 2-bromothiophene, 2-ethylthiophene, N-methylpyrrole and 2-bromopyridine may be mentioned.

C₁₋₄-Alkyl is a methyl, ethyl, propyl, butyl or isopropyl group.

The term “halogen atom” is understood in the context of the present invention as meaning a fluorine, chlorine, bromine or iodine atom; a fluorine, chlorine or bromine atom are preferred.

The above term “alkylheteroaromatic” is a heteroaromatic bonded to the ester function via a C₁₋₃-alkylene radical. Heteroaromatics are the groups named under heteroaromatic radical.

C₁₋₃-Alkylene radical is a methylene, ethylene or propylene bridge.

Preferred heteroaromatics are pyridine and thiophene.

Preferred compounds are listed below:
1) 3-hydroxyoestra-1,3,5(10)-trien-17β-yl 6′-sulphamoylnicotinate (1),
2) 3-hydroxyoestra-1,3,5(10)-trien-17β-yl 5′-sulph-amoylnicotinate (2),
3) 3-hydroxyoestra-1,3,5(10)-trien-17β-yl 2′-ethyl-5′-sulphamoylthiophene-3′-carboxylate (3),
4) 3-hydroxyoestra-1,3,5(10)-trien-17β-yl 2′-bromo-5′-sulphamoylthiophene-3′-carboxylate (4),
5) 3-oxoandrost-4-en-17β-yl 6′-sulphamoylnicotinate (5),
6) 3-oxoandrost-4-en-17β-yl 5′-sulphamoylnicotinate (6),
7) 3-oxoandrost-4-en-17β-yl 2′-ethyl-5′-sulphamoylthiophene-3′-carboxylate,
8) 3-oxoandrost-4-en-17β-yl 5′-sulphamoylthiophene-3′-carboxylate,
9) 3-hydroxyoestra-1,3,5(10)-trien-17β-yl 5′-sulph-amoylthiophene-3′-carboxylate,
10) 3-hydroxyoestra-1,3,5(10)-trien-17β-yl 6′-sulph-amoylthiophene-3′-carboxylate,
11) 3-oxoandrost-4-en-17β-yl 5′-sulphamoylthiophene-3′-carboxylate,
12) 3-oxo-7α-methylandrost-4-en-17β-yl 5′-sulphamoyl-nicotinate,
13) 3-oxo-7α-methylandrost-4-en-17β-yl 6′-sulphamoyl-nicotinate,
14) 3-oxo-7α-methylandrost-4-en-17β-yl N-ethyl-5′-sulphamoylthiophene-3′-carboxylate,
15) 3-hydroxyoestra-1,3,5(10)-trien-17β-yl N-methyl-5′-sulphamoyl-1H-pyrrole-2′-carboxylate.

The therapeutically relevant drug compound is released from the compounds according to the invention by hydrolysis.

In Vitro Experiments

a) Carboanhydrase Inhibition

Test Principle:

Photometric determination of the inhibition of human carboanhydrase I or II by sulphonamides or sulphamates on microtitre plates with the aid of the enzymatic conversion of nitrophenyl acetate with a colour change from colourless to yellow.

TABLE 1
IC₅₀ inhibitory values of human carboanhydrase I

| | CA II | CA I |
|---|---|---|
| Inhibitor | IC₅₀ (nM) | IC₅₀ (nM) |
| Oestradiol 3-sulphamate | 34 | 157 ± 10.6 |
| 3-hydroxyoestra-1,3,5(10)- | 31 | 2900 |
| trien-17β-yl 6′- | | |
| sulphamoylnicotinate (1) | | |
| 3-hydroxyoestra-1,3,5(10)- | 160 | 2100 |
| trien-17β-yl 5′- | | |
| sulphamoylnicotinate (2) | | |
| 3-oxoandrost-4-en-17β-yl 5′- | 250 | 2900 |
| sulphamoylnicotinate (6) | | |
| 3-oxoandrost-4-en-17β-yl 6′- | 41 | 3200 |
| sulphamoylnicotinate (5) | | |
| 3-hydroxyoestra-1,3,5(10)- | 42 | 3400 |
| trien-17β-yl 2′-ethyl-5′- | | |
| sulphamoylthiophene-3′- | | |
| carboxylate (3) | | |
| 3-hydroxyoestra-1,3,5(10)- | 47 | 3300 |
| trien-17β-yl 2′-bromo-5′- | | |
| sulphamoylthiophene-3′- | | |
| carboxylate (4) | | |
| Acetazolamide | 61 | 1200 |
| (known CA inhibitor) | | 1900¹⁾ |

Literature: ¹⁾C. Landolfi, M. Marchetti, G. Ciocci, and C. Milanese, Journal of Pharmacological and Toxicological Methods 38, 169-172 (1997).

It was found that the sulphamoyl prodrugs of the general formula (I) according to the invention inhibit carboanhydrase II surprisingly well. From this, a concentration of the prodrugs according to the invention in the erythrocytes can be derived.

b) Blood-plasma concentration ratio—test principle and experimental description:

The SO₂—NH₂ group of the substances according to the invention can lead to a concentration in erythrocytes as a result of binding to carboanhydrases.

Test Principle:

Freshly obtained, heparinized blood from a rat is treated with a defined amount of active compound. The active compound concentration in the plasma obtained therefrom is measured against a calibration curve of plasma which is spiked (with a known active compound concentration). The blood-plasma ratio is calculated from the measured concentration and the theoretical concentration.

TABLE 2
Blood/plasma ratio of selected prodrugs.

| | Blood/plasma |
|---|---|
| Prodrug | ratio |
| Oestradiol 3-sulphamate | About 50 |
| 3-hydroxyoestra-1,3,5(10)-trien- | 2 |
| 17β-yl 5′-sulphamoylnicotinate (2) | |
| 3-oxoandrost-4-en-17β-yl 5′- | 5.7 |
| sulphamoylnicotinate (6) | |
| 3-oxoandrost-4-en-17β-yl 6′- | 3.6 |
| sulphamoylnicotinate (5) | |
| 3-hydroxyoestra-1,3,5(10)-trien- | 1.8 |
| 17β-yl 2′-ethyl-5′- | |
| sulphamoylthiophene-3′- | |
| carboxylate (3) | |
| 3-hydroxyoestra-1,3,5(10)-trien- | 9.5 |
| 17β-yl 2′-bromo-5′- | |
| sulphamoylthiophene-3′- | |
| carboxylate (4) | |

In contrast to the results published in WO 01/91797, the concentration ratios of the compounds according to the invention between erythrocytes and plasma are not in a range from 10-1000:1, but in the range <10:1. This has shown itself as a crucial disadvantage for achieving therapy-relevant active compound levels. It is possible by the choice of suitable linkers to adjust the optimum blood/plasma ratio for a prodrug compound.

These test results open up to the compounds of the general formula (I) according to the invention, depending on the definition of “drug”, various possibilities for the treatment and/or prophylaxis of different syndromes. For example, for the case where “drug” is a steroid such as an androgen or oestrogen, the compounds of the general formula (I) can be used in hormone replacement therapy (HRT) in women and in men or in the treatment of hormonally caused diseases in men (carcinoma of the prostate, mammary carcinoma, hypogonadism) and women (endometriosis, mammary carcinoma). Furthermore, the compounds of the general formula (I) according to the invention, in which “drug” is, for example, an androgen or oestrogen, are used for fertility control in men or women.

The use of further active compounds mentioned for “drug” such as quinine, chinchonidine, hydroxychloroquine, primaquine or mefloquine concerns the treatment of malaria.

Compounds of the general formula (I) according to the invention in which “drug” is a cortisol derivative can be used for the treatment and prophylaxis of inflammatory and/or allergic diseases which are influenced by immunosuppressives and/or antiproliferatives.

Prodrugs according to the invention in which “drug” is a nucleoside (zidovudine, brivudine, indinavir, nelfinavir) can be employed for the treatment of viral diseases (herpes, HIV).

The invention moreover relates to the pharmaceutical compositions comprising the compounds of the general formula (I) according to the invention and optionally further active compounds, for example gestagens (norethisterone, dienogest, drospirenone, levonorgestrel), anti-gestagens (mifepristone, onapristone) and/or progesterone receptor modulators (mesoprogestins such as asoprisnil).

These pharmaceutical compositions and medicaments are preferably administered orally. In addition to customary vehicles and/or diluents, they contain at least one compound of the general formula I.

Dosage

The prodrugs according to the invention can be administered orally.

In general, satisfactory results are to be expected both for the treatment and/or prophylaxis of the indications mentioned or for fertility control if the dosage is carried out such that, after administration of the prodrugs, an amount of corresponding active compound (“drug”) is released which maximally corresponds to the highest dose of the respective “drug” substance used pharmaceutically.

The medicaments of the invention are prepared in a known manner with a suitable dosage using the customary solid or liquid vehicles or diluents and the pharmaceutical excipients customarily used according to the desired type of administration. The preferred preparations consist in an administration form which is suitable for oral administration. Such administration forms are, for example, tablets, film-coated tablets, coated tablets, capsules, pills, powders, solutions or suspensions or alternatively depot forms.

Appropriate tablets can be obtained, for example, by mixing the active compound with known excipients, for example inert diluents such as dextrose, sugar, sorbitol, mannitol, polyvinylpyrrolidone, disintegrants such as maize starch or alginic acid, binders such as starch or gelatine, lubricants such as magnesium stearate or talc and/or agents for achieving a depot effect such as carboxypolymethylene, carboxymethyl-cellulose, cellulose acetate phthalate or polyvinyl acetate. The tablets can also consist of a number of layers.

Correspondingly, coated tablets can be prepared by coating cores produced analogously to the tablets with agents customarily used in coated tablet coatings, for example polyvinylpyrrolidone or shellac, gum arabic, talc, titanium oxide or sugar. Here, the coated tablet shells can also consist of a number of layers, where the excipients mentioned above in the case of the tablets can be used.

Solutions or suspensions containing the compounds of the general formula I according to the invention can additionally comprise taste-enhancing agents such as saccharin, cyclamate or sugar and also, for example, flavourings such as vanillin or orange extract. They can moreover comprise suspending excipients such as sodium carboxymethylcellulose or preservatives such as p-hydroxybenzoates.

The capsules comprising compounds of the general formula I can, for example, be produced by mixing the compound(s) of the general formula I with an inert carrier such as lactose or sorbitol and encapsulating it (them) in gelatine capsules.

The prodrugs according to the invention can be synthesized according to the following examples, these serving for more detailed explanation, without restricting the invention.

The corresponding sulphamoylheteroarylcarboxylic acids are commercially obtainable or can be prepared from sulphonic acids or their derivatives by means of methods known to the person skilled in the art. Linkers which are not commercially obtainable can be synthesized as described by way of example below.

6-Sulphamoylnicotinic Acid

5 g of 6-thionicotinic acid are dissolved in 41 ml of conc. hydrochloric acid and 9 ml of water. The solution is cooled to 0-5° C. and chlorine is passed in for 4 h. Subsequently, the reaction solution is added to 100 g of ice, and the precipitated substance is filtered off and added to 100 ml of cold conc. NH₃ solution. After 1 h, the mixture is concentrated to 1/3 and acidified to pH=3 using HCl. The precipitated substance is filtered off with suction, washed with water and dried. Subsequently, purification is carried out by column chromatography. 6-Sulphamoylnicotinic acid is obtained.

¹H-NMR (DMSO-d₆): 7.62 (s, 2H, NH₂); 8.03 (m, 1H, CH); 8.50 (m, 1H, CH); 9.08 (m, 1H, CH).

5-Sulphamoylnicotinic Acid

1.) β-Picoline-5-sulphonic Acid

200 ml of oleum (25%) are initially introduced. 97 ml of β-picoline are added dropwise with cooling. 3.12 g of HgSO₄ are added at 40° C. and the mixture is subsequently heated at 225-235° C. for 16 h. About 100 ml of H₂SO₄ are then distilled off (160° C., 2 mbar). The mixture is then added to 400 g of ice and diluted with 2 l of water. It is subsequently neutralized using CaCO₃. The mixture is filtered off from inorganic constituents and the residue is washed with 2 l of boiling water. The aqueous solution is concentrated and the residue is chromatographed on silica gel. β-Picoline-5-sulphonic acid is obtained.

¹H-NMR (DMSO-d₆): 2.31 (s, 3H, CH₃); 7.75 (s, 1H, CH); 8.36 (s, 1H, CH); 8.57 (s, 1H, CH).

2.) 5-Sulphamoyl-β-picoline

3.5 g of β-picoline-5-sulphonic acid are mixed together with 6.5 g of PCl₅ and 2.5 ml of POCl₃ under protective gas and the mixture is heated at 120° C. for 3 h. The POCl₃ is distilled off in vacuo and 3 ml of ice water are added with cooling. The mixture is stirred into 150 ml of NH₃ soln. and the solution is concentrated to dryness. The residue is extracted with MeOH and the product obtained after concentration is purified by column chromatography on silica gel. 5-Sulphamoyl-β-picoline is obtained.

¹H-NMR (DMSO-d₆): 2.39 (s, 3H, CH₃); 7.56 (s, 2H, NH₂); 8.00 (s, 1H, CH); 8.62 (s, 1H, CH); 8.77 (s, 1H, CH).

3.) 5-Sulphamoylnicotinic Acid

8.0 g of 5-sulphamoyl-β-picoline are initially introduced in 250 ml of water. After addition of 12.5 g of KMnO₄, the mixture is warmed to 70° C. After decolourization, 12.5 g of KMnO₄ are added again and the mixture is warmed to 70° C. for 12 h. It is filtered hot and concentrated to about 20 ml. The residue is acidified (pH˜2) using 10% strength HCl. The substance crystallized in the cold is filtered off with suction, washed with water and dried. 5-Sulphamoylnicotinic acid is obtained.

¹H-NMR (DMSO-d₆): 7.76 (s, 2H, NH₂); 8.62 (m, 1H, CH); 9.15 (m, 1H, CH); 9.23 (m, 1H, CH).

### SYNTHESIS EXAMPLES

### Example 1

### 3-tert-Butyldimethylsilyloxyoestra-1,3,5(10)-trien-17β-yl 6′-sulphamoylnicotinate

0.5 g of 3-tert-butyldimethylsilyloxyoestra-1,3,5(10)-trien-17β-ol and 0.5 g of 6-sulphamoylnicotinic acid are dissolved in 7 ml of pyridine under argon. Subsequently, 0.1 g of p-Tos-OH and finally, at 0° C., 0.5 g of DCC are added. After 48 h, the reaction mixture is stirred at RT. For work-up, 40 ml of water are added and the mixture is adjusted to pH˜6 using 10% strength HCl. The precipitated substance is filtered off, washed with water and dried. It is purified by chromatography on silica gel. 3-tert-Butyldimethyl-silyloxyoestra-1,3,5(10)-trien-17β-yl 6′-sulphamoylnicotinate is obtained.

¹H-NMR (DMSO-d₆): 0.16 (s, 6H, SiMe), 0.938 (s, 9H, t-Bu), 0.944 (s, 3H, 18-Me), 4.90 (t, 1H, 17-H), 6.50-7.15 (3 m, 3H, CH_{Ar}), 7.69 (s, 2H, NH₂), 8.06, 8.55, 9.16 (3 m, 3H, CH_{Py}).

### Example 2

### 3-Hydroxyoestra-1,3,5(10)-trien-17β-yl 6′-sulphamoylnicotinate (1)

300 mg of 3-tert-butyldimethylsilyloxyoestra-1,3,5(10)-trien-17β-yl 6′-sulphamoylnicotinate are dissolved in 20 ml of THF. 250 mg of TBAF are added with stirring at RT. After 1 hour, 20 ml of water are stirred in. The substance is extracted with ethyl acetate. The organic phase is washed with satd. NaCl solution, dried over MgSO₄, filtered, concentrated and chromatographed on silica gel. 3-Hydroxyoestra-1,3,5(10)-trien-17β-yl 6′-sulphamoylnicotinate is obtained.

¹H-NMR (DMSO-d₆): 0.94 (s, 3H, 18-Me), 4.90 (t, 1H, 17-H), 6.40-7.15 (3 m, 3H, CH_{Ar}), 7.69 (s, 2H, NH₂); 8.06, 8.55 (2 m, 2H, CH_{Py}), 9.02 (s, 1H, 3-OH), 9.17 (1 s, 1H, CH_{Py}).

### Example 3

### 3-tert-Butyldimethylsilyloxyoestra-1,3,5(10)-trien-17β-yl 5′-sulphamoylnicotinate

0.55 g of 3-tert-butyldimethylsilyloxyoestra-1,3,5(10)-trien-17β-ol and 0.55 g of 5-sulphamoylnicotinic acid are dissolved in 7 ml of pyridine under argon. Subsequently, 0.12 g of p-Tos-OH and finally, at 0° C., 0.55 g of DCC are added. After 48 h, the reaction mixture is stirred at RT. For work-up, 40 ml of water are added and the mixture is adjusted to pH˜6 using 10% strength HCl. The precipitated substance is filtered off, washed with water and dried. It is purified by chromatography on silica gel. 3-tert-Butyldimethyl-silyloxyoestra-1,3,5(10)-trien-17β-yl 5′-sulphamoylnicotinate is obtained.

¹H-NMR (DMSO-d₆): 0.16 (s, 6H, SiMe), 0.94 (s, 9H, t-Bu), 0.95 (s, 3H, 18-Me), 4.92 (t, 1H, 17-H), 6.5-7.2 (3 m, 3H, CH_{Ar}), 7.79 (s, 2H, NH₂), 8.6-9.3 (3 m, 3H, CH_{Py}).

### Example 4

### 3-Hydroxyoestra-1,3,5(10)-trien-17β-yl 5′-sulphamoylnicotinate (2)

250 mg of 3-tert-butyldimethylsilyloxyoestra-1,3,5(10)-trien-17β-yl 5′-sulphamoylnicotinate are dissolved in 20 ml of THF. 220 mg of TBAF are added with stirring at RT. After 1 hour, 15 ml of water are stirred in. The substance is extracted with ethyl acetate. The organic phase is washed with satd. NaCl solution, dried over MgSO₄, filtered, concentrated and chromatographed on silica gel. 3-Hydroxyoestra-1,3,5(10)-trien-17β-yl 5′-sulphamoylnicotinate is obtained.

¹H-NMR (DMSO-d₆): 0.94 (s, 3H, 18-Me), 4.91 (t, 1H, 17-H), 6.4-7.1 (3 m, 3H, CH_{Ar}), 7.92 (s, 2H, NH₂); 8.61 (s, 1H, CH_{Py}), 9.00 (s, 1H, 3-OH), 9.17, 9.26 (2 s, 2H, CH_{Py}).

### Example 5

### 3-tert-Butyldimethylsilyloxyoestra-1,3,5(10)-trien-17β-yl 2′-ethyl-5′-sulphamoylthiophene-3′-carboxylate

0.75 g of 3-tert-butyldimethylsilyloxyoestra-1,3,5(10)-trien-17β-ol and 0.8 g of 5-(aminosulphonyl)-2-ethyl-3-thiophenecarboxylic acid are dissolved in 10 ml of pyridine under argon. Subsequently, 0.2 g of p-Tos-OH and finally, at 0° C., 0.75 g of DCC are added. After 48 h, the reaction mixture is stirred at RT. For work-up, 60 ml of water are added and the mixture is adjusted to pH˜6 using 10% strength HCl. The precipitated substance is filtered off, washed with water and dried. It is purified by chromatography on silica gel. 3-tert-Butyldimethylsilyloxyoestra-1,3,5(10)-trien-17β-yl 2′-ethyl-5′-sulphamoylthiophene-3′-carboxylate is obtained.

¹H-NMR (CDCl₃): 0.19 (s, 6H, SiMe), 0.92 (s, 3H, 18-Me), 0.97 (s, 9H, t-Bu), 1.34 (t, 3H, Et), 3.26 (q, 2H, Et), 4.86 (t, 1H, 17-H), 5.12 (s, 2H, NH₂), 6.50-7.15 (3 m, 3H, CH_{Ar}), 7.92 (s, 1H, CH_{Th}).

### Example 6

### 3-Hydroxyoestra-1,3,5(10)-trien-17β-yl 2′-ethyl-5′-sulphamoylthiophene-3′-carboxylate (3)

440 mg of 3-tert-butyldimethylsilyloxyoestra-1,3,5(10)-trien-17β-yl 2′-ethyl-5′-sulphamoylthiophene-3′-carboxylate are dissolved in 20 ml of THF. 400 mg of TBAF are added with stirring at RT. After 1 hour, 20 ml of water are stirred in. The reaction mixture is concentrated and the precipitated substance is filtered off with suction, washed with water and chromatographed on silica gel. 3-Hydroxyoestra-1,3,5(10)-trien-17β-yl 2′-ethyl-5′-sulphamoylthiophene-3′-carboxyiate (3) is obtained.

¹H-NMR (DMSO-d₆): 0.89 (s, 3H, 18-Me) 1.27 (t, 3H, Et), 3.20 (q, 2H, Et), 4.79 (t, 1H, 17-H), 6.35-7.05 (3 m, 3H, CH_{Ar}), 7.72 (s, 1H, CH_{Th}), 7.76 (s, 2H, NH₂), 9.01 (s, 1H, 3-OH).

### Example 7

### 3-tert-Butyldimethylsilyloxyoestra-1,3,5(10)-trien-17β-yl 2′-bromo-5′-sulphamoylthiophene-3′-carboxylate

0.75 g of 3-tert-butyldimethylsilyloxyoestra-1,3,5(10)-trien-17β-ol and 0.8 g of 5-(aminosulphonyl)-2-bromo-3-thiophenecarboxylic acid are dissolved in 10 ml of pyridine under argon. Subsequently, 0.2 g of p-Tos-OH and finally, at 0° C., 0.75 g of DCC are added. After 48 h, the reaction mixture is stirred at RT. For work-up, 70 ml of water are added and the mixture is adjusted to pH˜6 using 10% strength HCl. The precipitated substance is filtered off, washed with water and dried. It is purified by chromatography on silica gel. 3-tert-Butyldimethylsilyloxyoestra-1,3,5(10)-trien-17β-yl 2′-bromo-5′-sulphamoylthiophene-3′-carboxylate is obtained.

¹H-NMR (CDCl₃): 0.19 (s, 6H, SiMe), 0.94 (s, 3H, 18-Me), 0.97 (s, 9H, t-Bu), 4.88 (t, 1H, 17-H), 5.22 (s, 2H, NH₂), 6.50-7.10 (3 m, 3H, CH_{Ar}), 7.85 (s, 1H, CH_{Th}).

### Example 8

### 3-Hydroxyoestra-1,3,5(10)-trien-17β-yl 2′-bromo-5′-sulphamoylthiophene-3′-carboxylate (4)

330 mg of 3-tert-butyidimethylsilyloxyoestra-1,3,5(10)-trien-17β-yl 2′-bromo-5′-sulphamoyithiophene-3′-carboxylate are dissolved in 30 ml of THF. 300 mg of TBAF are added with stirring at RT. After 1 hour, 30 ml of water are stirred in. The reaction mixture is concentrated and the precipitated substance is filtered off with suction, washed with water and chromatographed on silica gel. 3-Hydroxyoestra-1,3,5(10)-trien-17β-yl 2′-bromo-5′-sulphamoylthiophene-3′-carboxylate (4) is obtained.

¹H-NMR (DMSO-d₆): 0.96 (s, 3H, 18-Me), 4.84 (t, 1H, 17-H), 6.40-7.15 (3 m, 3H, CH_{Ar}), 7.75 (s, 1H, CH_{Th}), 8.05 (s, 2H, NH₂), 9.01 (s, 1H, 3-OH).

### Example 9

### 3-Oxoandrost-4-en-17β-yl 6′-sulphamoylnicotinate (5)

0.4 g of testosterone is dissolved in 2 ml of pyridine. After addition of 0.4 g of 6-sulphamoylnicotinic acid, 50 mg of p-toluenesulphonic acid and 0.4 g of dicyclohexylcarbodiimide (DCC), the mixture is stirred at room temperature for 72 hours. Subsequently, 10 ml of water are added. The mixture is acidified slightly (pH=5) using 10% strength HCl. The precipitate is filtered off and washed 2× with satd. NaHCO₃ soln. and water. The dried residue is extracted with ethyl acetate. The organic phase is washed with 10% strength NaHCO₃ solution and satd. NaCl solution, dried over MGSO₄, filtered, concentrated and chromatographed on silica gel. 3-Oxoandrost-4-en-17β-yl 6′-sulphamoylnicotinate (5) is obtained.

¹H-NMR (DMSO-d₆): 0.95 (s, 3H, Me); 1.17 (s, 3H, Me); 5.64 (s, 1H, 4-H); 7.68 (s, 2H, NH₂); 8.06, 8.53, 9.15 (3 m, 3H, CH_{Ar}).

### Example 10

### 3-Oxoandrost-4-en-17β-yl 5′-sulphamoylnicotinate (6)

0.4 g of testosterone is dissolved in 2 ml of pyridine. After addition of 0.4 g of 5-sulphamoylnicotinic acid, 50 mg of p-toluenesulphonic acid and 0.4 g of dicyclohexylcarbodiimide (DCC), the mixture is stirred at room temperature for 72 hours. Subsequently, 10 ml of water are added. The mixture is slightly acidified (pH=5) using 10% strength HCl. The precipitate is filtered off and washed 2× with satd. NaHCO₃ soln. and water. The dried residue is extracted with ethyl acetate. The organic phase is washed with 10% strength NaHCO₃ solution and satd. NaCl solution, dried over MGSO₄, filtered, concentrated and chromatographed on silica gel. 3-Oxoandrost-4-en-17β-yl 5′-sulphamoylnicotinate (6) is obtained.

¹H-NMR (DMSO-d₆): 0.96 (s, 3H, Me); 1.17 (s, 3H, Me); 5.64 (s, 1H, 4-H); 7.76 (s, 2H, NH₂); 8.59, 9.17, 9.24 (3 s, 3H, CH_{Ar}).

### Example 11

### 3-tert-Butyldimethysilyloxyoestra-1,3,5(10)-trien-17β-yl N-methyl-5′-sulphamoyl-1H-pyrrole-2′-carboxylate

0.50 g of 3-tert-butyldimethylsilyloxyoestra-1,3,5(10)-trien-17β-ol and 0.50 g of N-methyl-5′-sulphamoyl-1H-pyrrole-2′-carboxylic acid are dissolved in 7 ml of pyridine under argon. Subsequently, 0.12 g of p-Tos-OH and finally, at 0° C., 0.5 g of DCC are added. After 48 h, the reaction mixture is stirred at RT. For work-up, 40 ml of water are added and the mixture is adjusted to pH˜6 using 10% strength HCl. The precipitated substance is filtered off, washed with water and dried. It is purified by chromatography on silica gel. 3-tert-Butyldimethylsilyloxyoestra-1,3,5(10)-trien-17β-yl N-methyl-5′-sulphamoyl-1H-pyrrole-2′-carboxylate is obtained.

¹H-NMR (CDCl₃): 0.18 (s, 6H, SiMe), 0.92 (s, 3H, 18-Me), °. 97 (s, 9H, t-Bu), 3.95 (s, 3H, NMe), 4.83 (t, 1H, 17-H), 4.95 (s, 2H, NH₂), 6.5-7.3 (5 m, 5H, CH_{Ar}).

### Example 12

### 3-Hydroxyoestra-1,3,5(10)-trien-17β-yl N-methyl-5′-sulphamoyl-1H-pyrrole-2′-carboxylate

300 mg of 3-tert-butyldimethylsilyloxyoestra-1,3,5(10)-trien-17β-yl N-methyl-5′-sulphamoyl-1H-pyrrole-2′-carboxylate are dissolved in 20 ml of THF. 250 mg of TBAF are added with stirring at RT. After 1 hour, 20 ml of water are stirred in. The reaction mixture is concentrated and the precipitated substance is filtered off with suction, washed with water and chromatographed on silica gel. 3-Hydroxyoestra-1,3,5(10)-trien-17β-yl N-methyl-5′-sulphamoyl-1H-pyrrole-2′-carboxylate is obtained.

¹H-NMR (DMSO-d₆): 0.89 (s, 3H, 18-Me), 3.88 (s, 3H, NMe), 4.76 (t, 1H, 17-H), 7.12 (s, 2H, NH₂), 8.99 (s, 1H, 3-OH).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2005 047 421.1, filed Nov. 30, 2005, and U.S. Provisional Application Ser. No. 60/742,523, filed Dec. 6, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Sulphonamide prodrugs of the general formula I 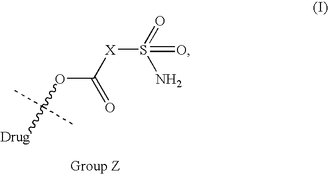
in which
X is an unsubstituted or substituted heteroaromatic radical or an alkylheteroaromatic and,
Drug is a pharmaceutical active compound which can form a carboxylic acid ester by means of an OH group, such as steroids, anti-malarial agents, nucleosides, isoflavanoids, which can optionally be substituted.

2. Sulphonamide prodrugs according to claim 1, where X is a pyridine or a thiophene radical.

3. Sulphamoylsulphonate prodrugs according to claim 1, where
Drug is steroids such as oestrogens, for example oestradiol or oestriol or androgens, for example testosterone, MENT (7α-methyl-19-nor-testosterone), eF-MENT (11-fluoro-7α-methyl-19-nortestosterone), nandrolone, DHT (dihydro-testosterone) or
gestagens, for example norethisterone, dienogest or levonorgestrel
corticoids, for example cortisol
anti-malarial agents, for example quinine, chinchonidine, hydroxychloroquine, primaquine, mefloquine or
nucleosides consisting of a sugar such as ribose or deoxyribose and of a base such as adenine, guanine, cytosine, thymine or uracil, furthermore
zidovudine, brivudine, indinavir, nelfinavir
isoflavanoids, for example genistein.

4. Sulphamoylsulphonate prodrugs according to claim 1, namely
1) 3-hydroxyoestra-1,3,5(10)-trien-17β-yl 6′-sulphamoylnicotinate (1),
2) 3-hydroxyoestra-1,3,5(10)-trien-17β-yl 5′-sulphamoylnicotinate (2),
3) 3-hydroxyoestra-1,3,5(10)-trien-17β-yl 2′-ethyl-5′-sulphamoylthiophene-3′-carboxylate (3),
4) 3-hydroxyoestra-1,3,5(10)-trien-17β-yl 2′-bromo-5′-sulphamoylthiophene-3′-carboxylate (4),
5) 3-oxoandrost-4-en-17β-yl 6′-sulphamoylnicotinate (5),
6) 3-oxoandrost-4-en-17β-yl 5′-sulphamoylnicotinate (6),
7) 3-oxoandrost-4-en-17β-yl 2′-ethyl-5′-sulphamoylthiophene-3′-carboxylate,
8) 3-oxoandrost-4-en-17β-yl 5′-sulphamoylthiophene-3′-carboxylate,
9) 3-hydroxyoestra-1,3,5(10)-trien-17β-yl 5′-sulphamoylthiophene-3′-carboxylate,
10) 3-hydroxyoestra-1,3,5(10)-trien-17β-yl 6′-sulphamoylthiophene-3′-carboxylate,
11) 3-oxoandrost-4-en-17β-yl 5′-sulphamoylthiophene-3′-carboxylate,
12) 3-oxo-7α-methylandrost-4-en-17β-yl 5′-sulph-amoylnicotinate,
13) 3-oxo-7α-methylandrost-4-en-17β-yl 6′-sulpha-moylnicotinate,
14) 3-oxo-7α-methylandrost-4-en-17β-yl N-ethyl-5′-sulphamoylthiophene-3′-carboxylate,
15) 3-hydroxyoestra-1,3,5(10)-trien-17β-yl N-methyl-5′-sulphamoyl-1H-pyrrole-2′-carboxylate.

5. Compounds according to claim 1, where the active compound is an antimalarial agent such as arteether, artemether, artesunate, chloroquine, pamaquine, primaquine, pyrethamine, mefloquine, proguanil, chinchonidine, cinchonine, hydroxychloroquine, pamaquine, primaquine, pyrimethamine, quinine or a quinine derivative, such as quinine bisulphate, quinine carbonate, quinine dihydrobromide, quinine dihydrochloride, quinine ethylcarbonate, quinine formate, quinine gluconate, quinine hydroiodide, quinine hydrochloride, quinine salicylate or quinine sulphate.

6. Use of the compounds according to claim 5 for the prevention of a parasitic attack on erythrocytes.

7. Compounds according to claim 1, where the therapeutically desired action takes place by release, in particular hydrolytic cleavage, of the active compound contained in the prodrug or its metabolites.

8. Pharmaceutical composition comprising at least one compound of the general formula I according to claim 1 and optionally at least one further active compound together with pharmaceutically tolerable excipients and/or vehicles.

9. Pharmaceutical composition according to claim 8, where the further active compound is a steroidal compound.

10. Pharmaceutical composition according to claim 9, where the further steroidal compound is a gestagen, anti-gestagen or a progesterone receptor modulator.

11. Pharmaceutical composition according to claim 10, in which the gestagens contained are norethisterone, dienogest, drospirenone, levo-norgestrel, the anti-gestagens mifepristone, onapristone and progesterone receptor modulators, for example mesoprogestins such as asoprisnil.

12. Use of compounds according to claim 1 for the production of a medicament.

13. Use according to claim 12 for production of a medicament for hormone replacement therapy.

14. Use of compounds according to claim 1 for female fertility control.

15. Use according to claim 12 for the production of a medicament for the therapy and/or prophylaxis of hormonally caused diseases in men and women.

16. Use according to claim 12 for the production of a medicament for the therapy and prophylaxis of endometriosis, mammary carcinomas, carcinomas of the prostate or hypogonadism.

17. Use according to claim 12 for the production of a medicament for the therapy and/or prophylaxis of diseases which can be positively influenced by the inhibition of the carboanhydrase activity.

18. Use according to claim 12 for the production of a medicament for the therapy and/or prophylaxis of inflammatory and/or allergic diseases.

19. Process for the preparation of the sulphonamide prodrugs of the general formula (I) according to claim 1 by reaction of a carboxylic acid NH₂SO₂—X—COOH of the corresponding heteroaromatic linker in the presence of dicyclohexylcarbodiimide or EDC (N-(3-dimethylaminopropyl)-N′-ethylcarbodiimide) optionally using a catalyst, with the appropriate active compound or by coupling of a sulphamoyl-carboxylic acid chloride of the corresponding heteroaromatic linker with the appropriate active compound in the presence of a base, preferably pyridine.

20. Process according to claim 19, where the base is pyridine.

21. Process according to claim 20, where the catalyst is p-toluenesulphonic acid.
